(19) United States
(12) Patent Application Publication
(10) Pub. No.: US 2020/0237901 A1
(43) Pub. Date: Jul. 30, 2020

(54) BIOACTIVE PREPARATIONS SPECIFIC AGAINST VARIOLA VIRUS INFECTION MODEL STRAINS AND APPLICATIONS THEREOF

(71) Applicant: Institute of Disease Prevention and Control, Academy of Militaary Medical Sciences, Beijing (CN)

(72) Inventors: Huanle LUO, Beijing (CN); Guohui Chang, Beijing (CN)

(21) Appl. No.: 15/761,760

(22) PCT Filed: Nov. 25, 2016
(86) PCT No.: PCT/CN2016/107230

§ 371 Date: Mar. 20, 2018

(30) Foreign Application Priority Data
Mar. 2, 2016 (CN) 201610118211.4

(51) Int. Cl. A61K 39/285; A61K 35/76; A61K 35/36

(52) U.S. Cl. CPC A61K 39/285; A61K 2039/552; A61K 35/36; A61K 35/76

(57) ABSTRACT

The present invention discloses a bioactive preparation specific against variola virus infection model strains and applications thereof. The application provided by the present invention specifically is the use of bioactive preparation CH2009 in following (1) or (2): (1) preparing the products for inhibiting poxvirus infection; or (2) using for inhibiting poxvirus infection. Bioactive preparation CH2009 is a bioactive preparation specific against poxvirus infection prepared and obtained from inflammatory skin tissues by intradermal vaccination of New Zealand white rabbit with vaccinia virus, and through the steps such as high temperature and pressure, solvent extraction, acid-base treatment, adsorption, elution, concentration by centrifugation and others. The present invention provides a completely new design strategy for the development of new generation of drugs against poxviridae infection such as smallpox and others, and it is no doubt that the resultant antiviral drugs have a wide application prospect and a huge commercial value.

### TECHNICAL FIELD

The present invention belongs to the field of antiviral drugs, and relates to bioactive preparations specific against variola virus infection model strains and applications thereof.

### BACKGROUND ART

Poxvirus is a huge family which can infect both human and animals, causing local or systemic suppurative skin lesions in infected subjects, which results in the outbreak of infectious diseases. Variola virus is especially notable since it rapidly breeds and can be transmitted through the air. Besides, the transmission rate is very fast, and the patients carrying such viruses are most infectious within one week after being infected due to a maximum amount of Variola virus in their saliva. Until the scabs of the patients are exfoliated, Variola virus still spread through patients, and it has lead to multiple global epidemic outbreaks in human history. In the case of malignant infection, Variola virus causes serious spreading damages in tissue layers or deeper layers of skin, and a large amount of patient's blood flow into skin, throat, lungs, intestines or uterus, resulting in uncontrollable toxemia or hemorrhoea. These patients exhibit symptoms including ordinary red, purple cicatrices, petechiae, and erythema morbilliforme rather than typical papulae or vesicular protuberances. Usually, the patients died within 10 to 14 days after the symptoms occurring, and lethal rate is up to 25-30%.

As extinction of smallpox and termination of vaccines worldwide, the human population susceptible to poxviruses such as smallpox and the like is increasing. Besides, the possibility for poxviruses being used as bioterrorism and potential biowarfare to human is more and more aggravated. Thus it is very important to launch the developments and resource reserve of drugs against Variola virus all around the world timely. Therefore, the developments of drugs against poxviridae such as smallpox and the like are always highly concerned by international community.

Because of the extreme dangers of Variola virus, the international community has made strict regulations, which greatly restrict the scientific researches to use Variola virus as research subject. Currently, vaccinia virus, monkey pox virus and Ectromelia virus infection model systems are used for replacing Variola virus infection, which is widely accepted by the international community, and with such Variola virus infection model systems, some compounds have been demonstrated to have inhibitory effects on various poxvirus infections, including Variola virus infection. These compounds comprise thiosemicarbazones, nucleosides and nucleoside analogues, interferons and interferon inducers, and the like. Currently, the substances, which are most widely studied and have a better inhibitory effect on vaccinia virus infection, are also mainly various nucleoside compounds and nucleoside analogues including inosine monophosphate (IMP) dehydrogenase inhibitors (e.g., ribavirin), S-adenosyl cysteine (SAH) hydrolase inhibitors (e.g., adenosine analogue A), orotidine monophosphate (OMP) decarboxylase inhibitors (e.g., pyrazofurin), cytidine triphosphate (CTP) synthase inhibitors (e.g., cyclopentenyl cytosine (Ce-Cyd)), acyclic nucleoside phosphonate compounds, such as cidofovir. However, such anti-poxvirus compounds show low bioavailable efficiency, large toxic and side effects, and so on, resulting in their comprehensive popularizations and applications are severely limited. Therefore, the developments of new safe and efficient drugs against poxviridae infections such as smallpox infection and the like are always highly concerned by international community.

### DISCLOSURE OF THE INVENTION

The objective of the present invention is to provide bioactive preparations specific against variola virus infection model strains and applications thereof.

The application provided by the present invention specifically is use of bioactive preparation CH2009 in following (1) or (2):

(1) preparing products for inhibiting poxvirus infection; or

(2) using for inhibiting poxvirus infection;

The bioactive preparation CH2009 can be specifically prepared and obtained according to a method comprising the following steps (a1)-(a6):

(a1) obtaining ex-vivo inflammatory skin of New Zealand white rabbit, placing it into 2% volume percent of phenol aqueous solution which is 3-4 folds (e.g., 3.5 folds) of the weight of obtained inflammatory skin, introducing nitrogen (e.g., 3 min), leaving for 60-80 h (e.g., 72 h) at 2-6° C. (e.g., 4° C.) after sealing, centrifuging (e.g., at 3000 rpm for 10 min), obtaining and filtering the supernate to obtain solution I (brown);

(a2) introducing nitrogen (e.g., 3 min) into the solution I, adjusting the pH of the solution I to 5.0±0.2 (e.g., 5.0) with 1 M hydrochloric acid, boiling in water bath for 25-35 min (e.g., 30 min), cooling down to 28±2° C. (e.g., 28° C.), centrifuging (e.g., at 3000 rpm for 10 min), obtaining and filtering the supernate to obtain solution II;

(a3) introducing nitrogen (e.g., 3 min) into the solution II, adjusting the pH of the solution II to 9.2±0.2 (e.g., 9.2) with 1 M sodium hydroxide, boiling in water bath for 25-35 min (e.g., 30 min), cooling down to 28±2° C. (e.g., 28° C.) and filtering, to obtain solution III;

(a4) introducing nitrogen (e.g., 3 min) into the solution III, adjusting the pH of the solution III to 4.5±0.2 (e.g., 4.5) with 1 M hydrochloric acid, introducing nitrogen (e.g., 3 min) into the solution III, adding activated carbon, continuously stirring under the condition of 30±2° C. (e.g., 30° C.) for 2-6 h (e.g., 4 h), resting for 25-35 min (e.g., 30 min) after the stirring is terminated, drawing and discarding the supernatant, filtering the remaining materials under nitrogen atmosphere, and then soaking and washing the activated carbon with physiological saline (pH 8.0), filtering under nitrogen atmosphere, discarding the filtrate, collecting and storing the activated carbon in a container, adding physiological saline (pH 8.0), adjusting the pH of the solution to 11.0±0.2 (e.g., 11.0) with 1 M sodium hydroxide, continuously stirring for 2-6 h (e.g., 4 h), filtering under nitrogen atmosphere, discarding the filtrate, and washing the activated carbon with physiological saline (pH 8.0), to obtain solution IV;

(a5) adjusting the pH of the solution IV to 6.0±0.2 (e.g., 6.0) with 1 M hydrochloric acid, sealing the solution IV within a container after nitrogen is introduced (e.g., 5 min after nitrogen is introduced), heating to 121±2° C. (e.g., 121° C.), maintaining for 15-25 min (e.g., 20 min), and then cooling down to below 40° C., to obtain solution V;

(a6) pumping the solution V into a reduced pressure distiller, and replacing air in the reduced pressure distiller with nitrogen, distillation under reduced pressure, filtering under the condition of 60±2° C. (e.g., 60° C.), the obtained filtrate is the bioactive preparation CH2009.

In the step (a1) of the method, the inflammatory skin of New Zealand white rabbit is the skin of New Zealand white rabbit, and the skin inflammation is induced by intradermal vaccination with poxvirus.

Here, the poxvirus may specifically be vaccinia virus (e.g., Vaccinia Virus WR Strain).

The vaccination amount of poxvirus is 5×10⁶-6×10⁶ TCID₅₀ per mature New Zealand white rabbit (2.7-3.0 kg in weight). The intradermal vaccination is a multiple spots intradermal vaccination, with 100-120 spots/rabbit. The New Zealand white rabbits were fed for 4-5 days after vaccination with the poxvirus (e.g., vaccinia virus), euthanatizing the New Zealand white rabbits, in which pox symptom was good, the color turned from red into purple red, the skin was thickened and there were subcutaneous edemas, and the collection of skin was completed within 15 min, the rabbit skin was packed with plastic bag, and was stored at −80° C. immediately for use.

In the step (a1) of the method, the filtration may be a filtration performed with a filter paper having a pore diameter of 2.5 μm (atmospheric pressure filtration). In step (a2), the filtration may be a filtration performed with a filter paper having a pore diameter of 2.5 μm (low pressure filtration). In step (a3), the filtration may be a filtration performed firstly with a filter paper having a pore diameter of 2.5 μm (low pressure filtration), and then with a filter membrane having a pore diameter of 0.45 μm (low pressure filtration). In step (a4), the first two filtrations both are filtrations performed with a filter paper having a pore diameter of 2.5 μm (low pressure filtration), and the third filtration may be a filtration performed with a filter membrane having a pore diameter of 0.45 μm (atmospheric pressure filtration). In step (a6), the filtration may be a filtration performed firstly with a filter paper having a pore diameter of 2.5 μm (atmospheric pressure filtration), and then with a filter membrane having a pore diameter of 0.2 μm (atmospheric pressure filtration).

In the method, the ratio among the weight of inflammatory skin in step (a1), the weight of activated carbon in step (a4), the volume of firstly used physiological saline in step (a4), the volume of secondly used physiological saline in step (a4), and the volume of liquid after reduced pressure distillation in step (a6) is about 200 g:40 g:400 ml:40 ml:5 ml.

In the method, the inhibition of poxvirus infection is following (b1) or (b2):

(b1) inhibiting the infection of poxvirus in host or host cells when simultaneously acting on the host or host cells together with poxvirus; or

(b2) treating poxvirus infection.

In (1) or (2) of the method, the poxvirus in the inhibition of poxvirus infection is a poxvirus which is able to infect mammals. The poxvirus which is able to infect mammals may specifically be Variola virus, vaccinia virus or Ectromelia virus. The vaccinia virus may specifically be Vaccinia Virus WR Strain. The Ectromelia virus may specifically be Ectromelia Virus Moscow strain.

The present invention also provides a product for inhibiting poxvirus infection.

The product for inhibiting poxvirus infection provided by the present invention is bioactive preparation CH2009, which is prepared and obtained according to the method comprising the above steps (a1)-(a6).

The inhibition of poxvirus infection is following (b1) or (b2):

(b1) inhibiting the infection of poxvirus in host or host cells when simultaneously acting on the host or host cells together with poxvirus; or

(b2) treating poxvirus infection.

The poxvirus is a poxvirus which is able to infect mammals. The poxvirus which is able to infect mammals may specifically be Variola virus, vaccinia virus or Ectromelia virus. The vaccinia virus may specifically be Vaccinia Virus WR Strain. The Ectromelia virus may specifically be Ectromelia Virus Moscow strain.

### BEST MODE OF THE INVENTION

Unless specially indicated, the experimental methods used in the following Examples are all conventional methods. Unless specially indicated, the materials, reagents and so on used in the following Examples are all commercially available.

Vaccinia Virus WR Strain: recorded in the article of “Sjoerd H. E. van den Worm, Klara Kristin Eriksson, Jessika C. Zevenhoven, Friedemann Weber, Roland Züst, Thomas Kuri, Ronald Dijkman, Guohui Chang, Stuart G. Siddell, Eric J. Snijder, Volker Thiel, Andrew D. Davidson. Reverse Genetics of SARS-Related Coronavirus Using Vaccinia Virus-Based Recombination. PLoS One. 2012; 7(3)”, which can be obtained from the applicant by the public, and is only used for repeating the experiments of the present invention.

Coronavirus (murine hepatitis coronavirus A59 strain, abbreviated as MHV-A59): recorded in the article of “Lin Lei, Sun Ying, Wu Xiaoyan, Sun Zounan, Yang Yi, Su Wenli, Hu Yi, Zhu Qingyu, Guo Deyin, Liu Jingmei, Chang Guohui. Attenuation of Mouse Hepatitis Virus by Deletion of the LLRK×G×KG Region of Nsp1. PLoS ONE, 2013 8(4):e61166.”, which can be obtained from the applicant by the public, and is only used for repeating the experiments of the present invention.

Ectromelia Virus Moscow strain (ECTV-MOS strain) (ATCC, VR-1374): recorded in the article of “Karolina Bien, Justyna Sokolowska, Piotr Bąska, Zuzanna Nowak, Wanda Stankiewicz and Malgorzata Krzyzowska. Fas/FasL Pathway Participates in Regulation of Antiviral and Inflammatory Response during Mousepox Infection of Lungs. Mediators Inflamm. 2015: 281613, doi: 10.1155/2015/281613;”, which can be obtained from the applicant by the public, and is only used for repeating the experiments of the present invention.

Adenovirus Type 5 (d1309 strain): recorded in the article of “Radko S, Jung R, Olanubi O, Pelka P. Effects of Adenovirus Type 5 E1A Isoforms on Viral Replication in Arrested Human Cells. PLoS One. 2015 Oct. 8; 10(10):e0140124. doi: 10.1371/journal.pone.0140124. eCollection 2015.”, which can be obtained from the applicant by the public, and is only used for repeating the experiments of the present invention.

Enterovirus 71 Hn2 strain (EV71-Hn2): recorded in the article of “Chang Guohui, Luo Yanjun, Wu Xiaoyan, Si Bingyin, Lin Lei and Zhu Qingyu. Monoclonal antibody induced with inactived EV71-Hn2 virus protects mice against lethal EV71-Hn2 virus infection. Virology Journal 2010, 7:106”, which can be obtained from the applicant by the public, and is only used for repeating the experiments of the present invention.

All of CV-1 cells, 293 cells and Vero cells are products from European Collection of Cell Cultures (http://www.ecacc.org.uk/), which are passage cultured according to conventional methods. 17Cl-1 cell is recorded in the article of “Lin Lei, Sun Ying, Wu Xiaoyan, Sun Zounan, Yang Yi, Su Wenli, Hu Yi, Zhu Qingyu, Guo Deyin, Liu Jingmei, Chang Guohui. Attenuation of Mouse Hepatitis Virus by Deletion of the LLRK×G×KG Region of Nsp1. PLoS ONE, 2013 8(4):e61166.”, being obtained from the applicant by the public, and is only used for repeating the experiments of the present invention. Each cell is passage cultured according to conventional methods.

Culture solution: A, cell growth culture solution: using DMEM culture medium (a product of Sigma company) as stock solution, adding 10% (volume fraction) fetal bovine serum (a product of invitrogen company), 0.2 mg/ml glutamine (a product of invitrogen company), 100 U/ml penicillin, streptomycin, respectively; B, cell maintaining culture solution, fetal bovine serum is 2% (volume fraction), the others are same as those in A; and C, virus proliferation culture solution: same as cell maintaining culture solution.

Animals: New Zealand white rabbits and Balb/c mice, SPF grade, both are products from Animal Research Center, Academy of Military Medical Sciences.

Cidofovir (Cidofovir hydrate): a product of Sigma company, CAS No.: 113852-37-2, Molecular Formula C₈H₁₄N₃O₆P.xH₂O, Molecular Weight 279.19, ≥98% (HPLC). It is dissolved in physiological saline at 37° C., and the concentration is formulated as 5 mg/ml.

Determination of virus titer: taking out 0.1 ml of viral stock cryopreserved at −80° C., adding it into a centrifuge tube containing 1 ml cell maintaining culture solution, that is, 10⁻¹ viral diluent, drawing 0.1 ml of viral diluent after uniform mixing with vibration, adding it into another centrifuge tube containing 1 ml cell maintaining solution, that is, 10⁻² viral diluent, and so on, to prepare 10-fold serially diluted viral diluents (10⁻¹, 10⁻², . . . , 10⁻¹²). Adding 0.1 ml of each viral diluent into 96-well cell culture plate in which the cells exhibited 80% confluent, infecting 4 wells in parallel with each viral dilution, and the plate was placed in incubator at 37° C., 5% CO₂, after 48 h culturing, observing and recording cytopathic effect (CPE) under a microscope, making statistic analysis on the situations of CPE, and calculating virus titer using Behrens-Karber method.

IgTCID₅₀=(percentage of cytopathic ratio above 50%−50%)/(percentage of cytopathic ratio above 50%−percentage of cytopathic ratio below 50%)×difference between log of dilutions+1 g (dilution of cytopathic ratio above 50%).

### Example 1, Preparation and Ingredient Identification of Bioactive Preparation CH2009

I, Preparation of Bioactive Preparation CH2009

1, Taking stimulative antigen (Vaccinia Virus WR Strain) out of −80° C. ultra-low temperature refrigerator, placing it in water bath at 37° C. for rapid dissolution prior to adding appropriate PBS, the viral solution for vaccination (titer:1×10⁶ TCID₅₀/ml) was prepared.

2, Picking healthy, mature New Zealand white rabbits with a weight of about 2.7-3.0 kg, cutting off back hair, wiping and sterilizing unhairing region with 75% alcohol of cotton balls, intradermally vaccinating 0.05 ml of the above viral solution/vaccination site, and a total of 100-120 sites/rabbit were vaccinated.

3, The New Zealand white rabbits were fed for 4-5 days after vaccination with virus. Rabbits was euthanatized, in which the pox symptom was good, the color turned from red into purple red, the skin was thickened and there were subcutaneous edemas. The collection of skin was completed within 15 min; the size of the rabbit skin is about 20×20 cm, and the weight is about 200 g. Subsequently, the rabbit skin was packed with plastic bag, and was stored in −80° C. freezer immediately for use.

4, The frozen rabbit skin having a size of about 20×20 cm was cut into small pieces of about 1×1 cm, and all of them were placed into 2% phenol aqueous solution (volume percent, that is, 100 volumes of solution contain 2 volumes of phenol and 98 volumes of water) which is 3.5 folds of the pieces themselves by weight (about 200 g×3.5=700 g), rapidly introducing nitrogen for 3 min, then leaving for 72 h at 4° C. after sealing, centrifuging at 3000 rpm for 10 min after the solution turned into emulsion, picking the supernate and filtering it with filter paper No. 5 (pore diameter: 2.5 μm) (atmospheric pressure), brown solution I was obtained.

5, Introducing nitrogen (e.g., 3 min) into solution I, adjusting the pH of solution I to 5.0 with 1 M hydrochloric acid, boiling in water bath for 30 min, cooling down to 28° C. immediately, centrifuging at 3000 rpm for 10 min prior to low pressure filtering with filter paper No. 5 (pore diameter: 2.5 μm), solution II was obtained.

6, Introducing nitrogen into solution II for 3 min, adjusting the pH of solution II to 9.2 with 1 M sodium hydroxide, boiling in water bath for 30 min, cooling down to 28° C. immediately, and low pressure filtering with filter paper No. 5 (pore diameter: 2.5 μm) and 0.45 μm filter membrane successively, solution III was obtained.

7, Introducing nitrogen into solution III for 3 min, adjusting the pH of solution III to 4.5 with 1 M hydrochloric acid, introducing nitrogen into solution III for 3 min, then adding 40 g activated carbon, continuously stirring under the condition of 30° C. for 4 h, resting for 30 min after the stirring was terminated, discarding the supernatant, low pressure filtering the remaining materials with filter paper No. 5 (pore diameter: 2.5 μm) under nitrogen atmosphere, and then soaking and washing the activated carbon with physiological saline for injection (pH 8.0), low pressure filtering with filter paper No. 5 (pore diameter: 2.5 μm) under nitrogen atmosphere, discarding the filtrate, collecting and storing the activated carbon in a container, adding 400 ml physiological saline for injection, adjusting the pH of the solution to 11.0 with 1 M sodium hydroxide, and continuously stirring for 4 h. Filtering with 0.45 μm filter membrane under nitrogen atmosphere (atmospheric pressure), and adequately washing the activated carbon with 40 ml physiological saline for injection, solution IV was obtained.

8, Adjusting the pH of solution IV to 6.0 with 1 M hydrochloric acid, sealing solution IV within a container 5 min after nitrogen was introduced (only needing to be full), heating to 121° C., maintaining for 20 min, and then cooling down to below 40° C., solution V was obtained.

9, Pumping solution V into a reduced pressure distiller, and replacing air in the reduced pressure distiller with nitrogen, reduced pressure distilling to a volume of 5 ml under the condition of 60° C., filtering with filter paper No. 5 (pore diameter: 2.5 μm) (atmospheric pressure), then filtering with 0.2 μm filter membrane (atmospheric pressure), a bioactive preparation about 5 ml was obtained, which is named as CH2009.

II, Ingredient Identification of Biological Preparation CH2009

1, Qualitative Detection of Proteins

Appropriate bioactive preparation CH2009 was taken, and protein qualitative detection was performed with coomassie brilliant blue method (Bradford method).

2, Qualitative Detection of Ingredients Such as Amino Acids, Polypeptides and the Like

Appropriate bioactive preparation CH2009 was taken, and amino acids and polypeptides were qualitatively detected using ninhydrin reaction.

3, Identification of Ingredients Such as Amino Acids, Polypeptides and the Like

Appropriate bioactive preparation CH2009 was taken, and kinds and contents of amino acids, nucleic acids and others were detected with liquid chromatography.

The results show that: bioactive preparation CH2009 is colorless or light yellow liquid, pH 7.5, and has ultraviolet absorption at 265-275 nm, with positive ninhydrin reaction, and negative protein detection. Detections of organic ingredients and contents were carried out for CH2009 with high performance liquid chromatography technique, and the results show that, this bioactive preparation CH2009 contains 13 kinds of amino acid compounds comprising aspartic acid, threonine, serine, glutamic acid, glycine, alanine, valine, isoleucine, leucine, tyrosine, phenylalanine, lysine and histidine, and also contains 5 kinds of nucleic acid compounds comprising urocanic acid, uracil, hypoxanthine, xanthine and thymine; the content (μg/ml) of each compound is: aspartic acid 0.3, threonine 0.2, serine 0.5, glutamic acid 1.1, glycine 0.5, alanine 0.6, valine 0.3, isoleucine 0.1, leucine 0.3, tyrosine 0.4, phenylalanine 0.2, lysine 0.1, histidine 0.2, urocanic acid 18.2, uracil 9.5, hypoxanthine 1.1, xanthine 8.6, thymine 2.0, respectively. It can be seen that biological preparation CH2009 is a biological extract containing a plurality of amino acids and nucleotides, and it necessarily has significant advantages such as low toxicity, high absorbility when used as an antiviral drug.

### Example 2, Detection of Anti-Poxvirus Activity of Bioactive Preparation CH2009

In this Example, the substituted viral models of Variola virus which are well known in the art, vaccinia virus and Ectromelia virus, were used as test poxviruses, and the antiviral activities of bioactive preparation CH2009 on poxviruses such as smallpox and others were studied at cellular and animal levels, respectively.

I, Determination of Activity of Inhibiting Poxvirus Infection In Vitro by Bioactive Preparation CH2009

Test viruses and the corresponding sensitive cells: Vaccinia Virus WR Strain, Ectromelia Virus Moscow strain (ECTV-MOS strain), coronavirus (murine hepatitis coronavirus A59 strain, abbreviated as MHV-A59), Adenovirus Type 5 (d1309 strain) and Enterovirus 71 Hn2 strain (EV71-Hn2). The sensitive cell line for vaccinia virus and Ectromelia virus is CV-1 cells; the sensitive cell line for adenovirus is 293 cells; the sensitive cell line for MHV is 17C1-1 cells; and the sensitive cell line for EV71 is Vero cells.

Selecting the 96-well culture plate in which the growth density of monolayer cells is about 80%, removing cell growth culture solution, successively taking 100 μl corresponding viral solutions which are 10-fold serially diluted, and subsequently, bioactive preparations CH2009 were added in following two manners, respectively:

A after adequately mixed with an equal volume of the solution of bioactive preparation CH2009, it was successively added into 96-well culture plate;

B 24 hours after virus infection, the culture medium was removed, and after adequately mixing 2×culture media with an equal volume of the solution of bioactive preparation CH2009, it was successively added into 96-well culture plate.

Finally, the plates were cultured in incubator for 48 hours. In each experiment, negative control group (not infected with viruses, without adding bioactive preparation CH2009) and positive control group (infected with an equal amount of viruses, but without adding bioactive preparation CH2009) were set up, and cells in plates were observed daily under inverted microscope. The cytopathy of the positive control group is defined as “++++”, and virus titers (TCID₅₀) of each group were calculated and recorded.

Meanwhile, cidofovir, approved as an anti-variola virus reserved drug by FDA of US, was set up as positive control drug, specifically a control in which bioactive preparation CH2009 was replaced with an equal volume of cidofovir having a concentration of 5 mg/ml.

The results showed that: the effects of bioactive preparation CH2009 on virus titer (TCID₅₀) were compared and analyzed with the cellular infection models of vaccinia virus, Ectromelia virus, coronavirus, enterovirus and adenovirus, by adding bioactive preparation CH2009 under two conditions, at the meantime of virus infection and 24 hours after virus infection, respectively. CPE caused by virus infection was observed and results showed that bioactive preparation CH2009 not only reduced the titers of vaccinia virus by 10^{3.3} and 10^{3.1} folds, respectively, but also could decrease the titers of Ectromelia virus by 10^{3.4} and 10^{3.0} folds, respectively. While the corresponding positive control drug, cidofovir, effectively reduced the titers of vaccinia virus and Ectromelia virus by 10^{4.3} and 10^{4.0} folds, as well as 10^{4.0} and 10^{3.8} folds, respectively. Under two conditions, bioactive preparation CH2009 had no effects on the titers of coronavirus, enterovirus and adenovirus, although positive control drug, cidofovir, exhibited a certain effect on the titers of adenovirus (the virus titers were reduced by 10^{1.4} and 10^{1.1} folds, respectively). Similar with CH2009, cidofovir showed no effects on the titers of coronavirus (MHV) or enterovirus (EV71). The results are shown in Table 1 in detail.

TABLE 1
Effects of inhibiting host cells infection with various viruses
by bioactive preparation CH2009

| | Virus Titer (TCID₅₀) | | | | | | | |
|---|---|---|---|---|---|---|---|---|
| | Biological Preparation | | | | | | | |
| | (CH2009) | | | | Cidofovir | | | |
| Group | A | B | C | D | A | B | C | D |
| vaccinia virus | 10^{5.2} | 10^{5.4} | 0 | 10^{8.5} | 10^{4.2} | 10^{4.5} | 0 | 10^{8.5} |
| Ectromelia virus | 10^{4.1} | 10^{4.5} | 0 | 10^{7.5} | 10^{3.5} | 10^{3.7} | 0 | 10^{7.5} |
| Adenovirus Type 5 | 10^{9.5} | 10^{9.6} | 0 | 10^{9.6} | 10^{8.2} | 10^{8.5} | 0 | 10^{9.6} |
| Enterovirus 71 | 10^{8.1} | 10^{8.2} | 0 | 10^{8.2} | 10^{8.0} | 10^{8.3} | 0 | 10^{8.2} |
| murine hepatitis | 10^{8.0} | 10^{8.1} | 0 | 10^{8.0} | 10^{8.1} | 10^{7.9} | 0 | 10^{8.0} |
| coronavirus | | | | | | | | |

Note:
A. administration at the meantime of virus infection; B. administration 24 hours after virus infection; C. negative control; and D. positive control.

According to the above experimental results, it can be concluded: bioactive preparation CH2009 can specifically and efficiently inhibit vaccinia virus and Ectromelia virus infections. Although the efficiency is slightly lower than cidofovir, it still can significantly reduce virus titer by more than 1000 times. Meanwhile, bioactive preparation CH2009 has no inhibition effect on coronavirus, enterovirus and adenovirus, indicating that bioactive preparation CH2009 does only have specific inhibition effect on poxviridae.

II, Analysis of Effect of Bioactive Preparation CH2009 Against Poxvirus Attacks In Vivo

Test viruses: Ectromelia Virus Moscow strain (ECTV-MOS strain).

1, Determination of LD₅₀ of Ectromelia Virus

3˜4-week-old female Balb/c mice were randomly divided into 6 groups (10 mice/group). The stock solution of Ectromelia virus was 10-fold serially diluted, each mouse was intraperitoneally vaccinated with 100 μl different dilution of Ectromelia virus successively. Infected mice were monitored daily for weight loss, clinical signs of diseases and death. The lethal dose 50% (LD₅₀) of Ectromelia virus was calculated with Behrens-Karber method, that is, the dose of Ectromelia virus which can cause half of the experimental mice death.

2, Analysis of Effect of Bioactive Preparation CH2009 Against Virus Attacks In Vivo

3˜4-week-old female Balb/c mice were randomly divided into 8 groups (15/mice/group). Each mouse was intraperitoneally vaccinated with 100 μl Ectromelia virus infection mixing solution containing 100 LD₅₀. Drugs administration was performed by terms of four modes: A. administration at the meantime of infection with virus, the administration dose of cidofovir is 15 mg/kg of weight, the volume is 200 μl, the administration dose of bioactive preparation CH2009 is 200 μl stock solution, once a day, 200 μl per time, intraperitoneal injection, for 3 weeks; B. administration 24 hours after infection with virus, the administration dose of cidofovir is 15 mg/kg of weight, the volume is 200 μl, the administration dose of bioactive preparation CH2009 is 200 μl stock solution, once a day, 200 μl per time, intraperitoneal injection, for 3 weeks; C. negative control, administration without virus attacks, the administration dose of cidofovir is 15 mg/kg of weight, the volume is 200 μl, the administration dose of bioactive preparation CH2009 is 200 μl stock solution, once a day, 200 μl per time, intraperitoneal injection, for 3 weeks; D. positive control, drug is replaced with PBS, and 100 LD₅₀ Ectromelia viruses were used for virus attacks. Infected mice were monitored daily for weight loss, clinical signs of diseases and death for five weeks. The survival rate and case-fatality rate of the mice were calculated, respectively.

The results showed that: using mouse model intraperitoneally infected with 100 LD₅₀ of Ectromelia virus, bioactive preparation CH2009 and control drug cidofovir were added at the same time of virus infection or 24 hours after virus infection. After observation for 5 weeks, the results demonstrated that all the mice attacked with virus exhibited varying degrees of clinical symptoms such as anorexia, weight loss, towering hair, poor mental state and the like during first two weeks. The mice in drug administration group got better after two weeks, while non-administration group infected with virus started to show serious symptoms such as hunchback, abrosia and the like, even death cases, eventually all died within one week. After 5 weeks, the statistical results showed that, under two administration conditions (“administration 24 hours after infection with virus” and “administration at the meantime of infection with virus”), the survival rates of virus-attacking-mice injected with bioactive preparation CH2009 are 66.67% and 80%, respectively, the survival rates of virus-attacking-mice which were injected with control drug, cidofovir, are 86.67% and 93.33%, respectively, while all the mice infected with virus without drug administration died. The results are shown in Table 2 in detail. Although the protective effect of bioactive preparation CH2009 on mice is slightly lower than that of control drug, cidofovir, the protective effect above 50% still shows a highly potential application value.

TABLE 2
Protective effect of bioactive preparation CH2009 on mice attacked with virus

| | Biological Preparation (CH2009) | | | | Cidofovir | | | |
|---|---|---|---|---|---|---|---|---|
| | Survival | Death | Survival | Case-Fatality | Survival | Death | Survival | Case-Fatality |
| Group | Number | Number | Rate (%) | Rate (%) | Number | Number | Rate (%) | Rate (%) |
| A | 12 | 3 | 80 | 20 | 14 | 1 | 93.33 | 6.67 |
| B | 10 | 5 | 66.67 | 33.33 | 13 | 2 | 86.67 | 13.33 |
| C | 15 | 0 | 100 | 0 | 15 | 0 | 100 | 0 |
| D | 0 | 15 | 0 | 100 | 0 | 15 | 0 | 100 |

Note:
A. administration at the meantime of virus infection; B. administration 24 hours after virus infection; C. negative control; and D. positive control.

The results of this Example show that: bioactive preparation CH2009 shows specific inhibition effect on vaccinia virus, Ectromelia virus, and other poxviridae at cellular and animal levels, and although the antiviral efficiency is slightly lower than cidofovir, bioactive preparation CH2009 is a biological extract containing a plurality of amino acids and nucleotides, and has significant advantages such as low toxicity, high absorbility and others. Moreover, the effect of its resistance to various poxviridae including Variola virus will certainly be greatly enhanced after further concentration and purification. Therefore, it has extremely high commercial value as the only bioactive material against poxviridae such as smallpox and others in the world now.

### INDUSTRIAL APPLICATION

The present invention intradermally vaccinates New Zealand white rabbits with vaccinia viruses, and specific bioactive preparation CH2009 against poxvirus infection is prepared from the inflammatory skin tissues of the white rabbits via steps such as high temperature and pressure, solvent extraction, acid-base treatment, adsorption, elution, concentration by centrifugation and others. Biological preparation CH2009 is a biological extract containing a plurality of amino acids and nucleotides, and has significant advantages such as low toxicity, high absorbility and others. Moreover, the effect of its resistance to various poxviridae including Variola virus will certainly be greatly enhanced after further concentration and purification. Therefore, the present invention provides a completely new design strategy for the development of new generation of drugs against poxviridae infection such as smallpox and others, and it is no doubt that the resultant antiviral drugs have a wide application prospect and a huge commercial value.

1. A method of using a bioactive preparation CH2009 in the manufacture of products for inhibiting poxvirus infection wherein the bioactive preparation CH2009 is prepared according to a method comprising the following steps (a1)-(a6):
(a1) obtaining ex-vivo inflammatory skin of New Zealand white rabbit, placing the ex-vivo inflammatory skin into a 2% volume percent of phenol aqueous solution which is 3-4 fold a weight of the obtained ex-vivo inflammatory skin, introducing nitrogen, leaving for 60-80 hours at 2-6° C. after sealing, centrifuging, obtaining and filtering a supernate to obtain solution I;
(a2) introducing nitrogen into the solution I, adjusting a pH of the solution I to 5.0±0.2, boiling the solution I in a water bath for 25-35 min, cooling the solution I down to 28±2° C., centrifuging the solution I, obtaining and filtering a supernate of the solution I to obtain solution II;
(a3) introducing nitrogen into the solution II, adjusting a pH of the solution II to 9.2±0.2, boiling the solution II in the water bath for 25-35 min, cooling the solution II down to 28±2° C., and filtering the solution II, to obtain solution III;
(a4) introducing nitrogen into the solution III, adjusting a pH of the solution III to 4.5±0.2, introducing nitrogen into the solution III, adding activated carbon, continuously stirring the solution III and the activated charcoal under a condition of 30±2° C. for 2-6 hours, resting a mixture of the activated charcoal and solution III for 25-35 min after the stirring is terminated, drawing and discarding the supernatant of the mixture of the activated charcoal and solution III, performing a first filtration of remaining materials under nitrogen atmosphere, and then soaking and washing the activated carbon with a first volume of physiological saline, performing a second filtration under nitrogen atmosphere, discarding a filtrate, collecting and storing the activated carbon in a container, adding a second volume of physiological saline to the container containing the activated carbon, adjusting a pH of the solution to 11.0±0.2, continuously stirring for 2-6 hours, performing a third filtration under nitrogen atmosphere, discarding the filtrate, and washing the activated carbon with a third volume of physiological saline, to obtain solution IV;
(a5) adjusting a pH of the solution IV to 6.0±0.2, sealing the solution IV within a container after nitrogen is introduced, heating to 121±2° C., maintaining for 15-25 min, and then cooling down to below 40° C., to obtain a solution V;
(a6) pumping the solution V into a reduced pressure distiller, and replacing air in the reduced pressure distiller with nitrogen, distillation under reduced pressure, filtering under a condition of 60±2° C. to obtain a filtrate, wherein the obtained filtrate is the bioactive preparation CH2009.

2. The method of claim 1, wherein in step (a1), the inflammatory skin of New Zealand white rabbit is the skin of New Zealand white rabbit in which skin inflammation is induced by intradermal vaccination with poxvirus.

3. The method of claim 2, wherein the poxvirus is vaccinia virus.

4. The method of claim 2, wherein the vaccination amount of poxvirus is 5×10⁶-6×10⁶ TCID₅₀ per New Zealand white rabbit.

5. The method of claim 1, wherein
in step (a1), the filtration is a filtration performed with a filter paper having a pore diameter of 2.5 μm;
in step (a2), the filtration is a filtration performed with a filter paper having a pore diameter of 2.5 μm;
in step (a3), the filtration is a filtration performed firstly with a filter paper having a pore diameter of 2.5 μm, and then with a filter membrane having a pore diameter of 0.45 μm;
in step (a4), the first two filtrations both are filtrations performed with a filter paper having a pore diameter of 2.5 μm, and the third filtration is a filtration performed with a filter membrane having a pore diameter of 0.45 μm; and
in step (a6), the filtration is a filtration performed firstly with a filter paper having a pore diameter of 2.5 μm, and then with a filter membrane having a pore diameter of 0.2 μm.

6. The method of claim 1, wherein a ratio among the weight of inflammatory skin in step (a1), a weight of activated carbon in step (a4), a first volume of physiological saline in step (a4), a second volume of physiological saline in step (a4), and a volume of liquid after reduced pressure distillation in step (a6) is 200 g:40 g:400 ml:40 ml:5 ml.

7. The method of claim 1, wherein the inhibition of poxvirus infection is following (b1) or (b2):
(b1) inhibiting the infections of poxvirus in host or host cells when simultaneously acting on the host or host cells together with poxvirus; or
(b2) treating poxvirus infection.

8. The method of claim 1, wherein the poxvirus in the inhibition of poxvirus infection is a poxvirus which is able to infect mammals.

9. The method of claim 8, wherein the poxvirus which is able to infect mammals is Variola virus, vaccinia virus or Ectromelia virus.

10. A method of using a bioactive preparation CH2009 in inhibition of poxvirus infection wherein
the bioactive preparation CH2009 is prepared and obtained according to a method comprising the following steps (a1)-(a6):
(a1) obtaining ex-vivo inflammatory skin of New Zealand white rabbit, placing the ex-vivo inflammatory skin into a 2% volume percent of phenol aqueous solution which is 3-4 folds of a weight of the obtained ex-vivo inflammatory skin, introducing nitrogen, leaving for 60-80 hours at 2-6° C. after sealing, centrifuging, obtaining and filtering a supernate to obtain solution I;
(a2) introducing nitrogen into the solution I, adjusting a pH of the solution I to 5.0±0.2, boiling in a water bath for 25-35 min, cooling down to 28±2° C., centrifuging, obtaining and filtering a supernate to obtain solution II;
(a3) introducing nitrogen into the solution II, adjusting a pH of the solution II to 9.2±0.2, boiling in the water bath for 25-35 min, cooling down to 28±2° C., and filtering, to obtain solution III;
(a4) introducing nitrogen into the solution III, adjusting a pH of the solution III to 4.5±0.2, introducing nitrogen into the solution III, adding activated carbon, continuously stirring under a condition of 30±2° C. for 2-6 hours, resting for 25-35 min after the stirring is terminated, drawing and discarding the supernatant, performing a first filtration of remaining materials under nitrogen atmosphere, and then soaking and washing the activated carbon with a first volume of physiological saline, performing a second filtration under nitrogen atmosphere, discarding the filtrate, collecting and storing the activated carbon in a container, adding a second volume of physiological saline, adjusting a pH of the solution to 11.0±0.2, continuously stirring for 2-6 hours, performing a third filtration under nitrogen atmosphere, discarding the filtrate, and washing the activated carbon with a third volume of physiological saline, to obtain solution IV;
(a5) adjusting a pH of the solution IV to 6.0±0.2, sealing the solution IV within a container after nitrogen is introduced, heating to 121±2° C., maintaining for 15-25 min, and then cooling down to below 40° C., to obtain solution V; and
(a6) pumping the solution V into a reduced pressure distiller, and replacing air in the reduced pressure distiller with nitrogen, distillation under reduced pressure, filtering under a condition of 60±2° C., to obtain a filtrate, wherein the obtained filtrate is the bioactive preparation CH2009.

11. The method of claim 10, wherein in step (a1), the inflammatory skin of New Zealand white rabbit is the skin of New Zealand white rabbit in which skin inflammation is induced by intradermal vaccination with poxvirus.

12. The method of claim 11, wherein the poxvirus is vaccinia virus.

13. The method of claim 11, wherein the vaccination amount of poxvirus is 5×10⁶-6×10⁶ TCID₅₀ per New Zealand white rabbit.

14. The method of claim 10, wherein
in step (a1), the filtration is a filtration performed with a filter paper having a pore diameter of 2.5 μm;
in step (a2), the filtration is a filtration performed with a filter paper having a pore diameter of 2.5 μm;
in step (a3), the filtration is a filtration performed firstly with a filter paper having a pore diameter of 2.5 μm, and then with a filter membrane having a pore diameter of 0.45 μm;
in step (a4), the first two filtrations both are filtrations performed with a filter paper having a pore diameter of 2.5 μm, and the third filtration is a filtration performed with a filter membrane having a pore diameter of 0.45 μm;
in step (a6), the filtration is a filtration performed firstly with a filter paper having a pore diameter of 2.5 μm, and then with a filter membrane having a pore diameter of 0.2 μm.

15. The method of claim 10, wherein a ratio among the weight of inflammatory skin in step (a1), a weight of activated carbon in step (a4), a first volume of physiological saline in step (a4), a second volume of physiological saline in step (a4), and the volume of liquid after reduced pressure distillation in step (a6) is 200 g:40 g:400 ml:40 ml:5 ml.

16. The method of claim 10, wherein the inhibition of poxvirus infection is following (b1) or (b2):
(b1) inhibiting the infections of poxvirus in host or host cells when simultaneously acting on the host or host cells together with poxvirus; or
(b2) treating poxvirus infection.

17. The method of claim 10, wherein the poxvirus in the inhibition of poxvirus infection is a poxvirus which is able to infect mammals.

18. The method of claim 17, wherein the poxvirus which is able to infect mammals is Variola virus, vaccinia virus or Ectromelia virus.

19. A product for inhibiting poxvirus infection comprising a bioactive preparation CH2009 wherein
the bioactive preparation CH2009 is prepared and obtained according to a method comprising the following steps (a1)-(a6):
(a1) obtaining ex-vivo inflammatory skin of New Zealand white rabbit, placing it into 2% volume percent of phenol aqueous solution which is 3-4 folds of a weight of the obtained ex-vivo inflammatory skin, introducing nitrogen, leaving for 60-80 h at 2-6° C. after sealing, centrifuging, obtaining and filtering a supernate to obtain solution I;
(a2) introducing nitrogen into the solution I, adjusting a pH of the solution I to 5.0±0.2, boiling in a water bath for 25-35 min, cooling down to 28±2° C., centrifuging, obtaining and filtering a supernate to obtain solution II;
(a3) introducing nitrogen into the solution II, adjusting a pH of the solution II to 9.2±0.2, boiling in the water bath for 25-35 min, cooling down to 28±2° C., and filtering, to obtain solution III;
(a4) introducing nitrogen into the solution III, adjusting a pH of the solution III to 4.5±0.2, introducing nitrogen into the solution III, adding activated carbon, continuously stirring under a condition of 30±2° C. for 2-6 h, resting for 25-35 min after the stirring is terminated, drawing and discarding the supernatant, performing a first filtration of remaining materials under nitrogen atmosphere, and then soaking and washing the activated carbon with a first volume of physiological saline, performing a second filtration under nitrogen atmosphere, discarding the filtrate, collecting and storing the activated carbon in a container, adding a second volume of physiological saline, adjusting a pH of the solution to 11.0±0.2, continuously stirring for 2-6 h, performing a third filtration under nitrogen atmosphere, discarding the filtrate, and washing the activated carbon with a third volume of physiological saline, to obtain solution IV;
(a5) adjusting a pH of the solution IV to 6.0±0.2, sealing the solution IV within a container after nitrogen is introduced, heating to 121±2° C., maintaining for 15-25 min, and then cooling down to below 40° C., to obtain solution V;BU
(a6) pumping the solution V into a reduced pressure distiller, and replacing air in the reduced pressure distiller with nitrogen, distillation under reduced pressure, filtering under a condition of 60±2° C., the obtained filtrate is the bioactive preparation CH2009.

20. The product of claim 19, wherein in step (a1) of preparing preparation CH2009, the inflammatory skin of New Zealand white rabbit is the skin of New Zealand white rabbit in which skin inflammation is induced by intradermal vaccination with poxvirus.

21. The product of claim 20, wherein the poxvirus used in preparing the preparation CH2009 is vaccinia virus.

22. The product of claim 20, wherein the vaccination amount of poxvirus used in preparing the preparation CH2009 is 5×10⁶-6×10⁶ TCID₅₀ per New Zealand white rabbit.

23. The product of claim 19, wherein in the method of preparing preparation CH2009,
in step (a1), the filtration is a filtration performed with a filter paper having a pore diameter of 2.5 μm;
in step (a2), the filtration is a filtration performed with a filter paper having a pore diameter of 2.5 μm;
in step (a3), the filtration is a filtration performed firstly with a filter paper having a pore diameter of 2.5 μm, and then with a filter membrane having a pore diameter of 0.45 μm;
in step (a4), the first two filtrations both are filtrations performed with a filter paper having a pore diameter of 2.5 μm, and the third filtration is a filtration performed with a filter membrane having a pore diameter of 0.45 μm;
in step (a6), the filtration is a filtration performed firstly with a filter paper having a pore diameter of 2.5 μm, and then with a filter membrane having a pore diameter of 0.2 μm.

24. The product of claim 19, wherein in the preparation of bioactive preparation CH2009 a ratio among the weight of inflammatory skin in step (a1), a weight of activated carbon in step (a4), the first volume of physiological saline in step (a4), the second volume of physiological saline in step (a4), and the volume of liquid after reduced pressure distillation in step (a6) is 200 g:40 g:400 ml:40 ml:5 ml.

25. The product of claim 19, wherein the inhibition of poxvirus infection is following (b1) or (b2):
(b1) inhibiting the infections of poxvirus in host or host cells when simultaneously acting on the host or host cells together with poxvirus; or
(b2) treating poxvirus infection.

26. The product of claim 25, wherein: the poxvirus in the inhibition of poxvirus infection is a poxvirus which is able to infect mammals.

27. The product of claim 26, wherein the poxvirus which is able to infect mammals is Variola virus, vaccinia virus or Ectromelia virus.
